(19) United States
(12) Patent Application Publication
(10) Pub. No.: US 2002/0076359 A1
(43) Pub. Date: Jun. 20, 2002

(54) Chemical composition used as an air filter and method of use

(76) Inventors: Cathy D. Santa Cruz, Reno, NV (US); Stan Gittelman, Carson City, NV (US)

Correspondence Address: Cathy D. Santa Cruz, 7630 Tholl Drive, Reno, NV, 89506, US

(21) Appl. No.: 09/739,556

(22) Filed: Dec. 16, 2000

(51) Int. Cl. A61L 9/00; B32B 5/02; A62B 7/08; B32B 27/12; B32B 27/04

(52) U.S. Cl. 422/120; 422/5

(57) ABSTRACT

The present invention is substantially an air filter that is composed of an air permeable container having a special chemical composition therein, and can be used in any area wherein the environmental air is to be freshened. Also, this invention is unique because the chemical composition can be used for soil aeration purposes for delivering oxygen to the roots of plants after it has been used as an air filter.

### FIELD OF THE INVENTION

This invention relates to air filters in general but more specifically pertains to an air filter that is made from a specific chemical composition including a container having a filter bag therein for proper dispensing of the chemical therefrom. Also, the air filter is not limited to any specific type of use, as it is most versatile and can be used for freshening fowl air resultant from substantially anything that interferes with the normal fresh air environment.

### BACKGROUND OF THE INVENTION

Within the known prior art, numerous attempts have been proposed for creating an air filter made from various chemical compositions. For example, U.S. Pat. No. 5,468,447 entitled “REFRIGERATOR FRESHENER” teaches a freshening device including an air-permeable container. With the container enclosing a mixture in particulate form, which is made from a major amount of activated carbon and a minor amount of an odor-absorbing zeolite, such as 75 to 95 percent carbon and 5 to 25 percent zeolite. This air freshener is useful for its intended use as a refrigerator freshener but it is strictly limited to use within a refrigerator and is not usable for other purposes, unlike the present invention which offers other benefits inside a refrigerator in addition to odor control and can also be used in a multitude of applications for air freshening, or air filtering.

Other types of known prior art include U.S. Pat. No. 5,224,975 entitled “LITTER BOX DEODORIZER” and U.S. Pat. No. 4,146,566 entitled “SELF-CONTAINED VOLATILE COMPONENT DISPENSING PACKAGE”. Each of which are somewhat functional for their intended use but each are limited and the chemical compositions are not as effective for freshening the air as is the chemical composition of the present invention.

Further prior art includes many different types of products such as mists, blocks, liquids, powders, granules and gases. However, each have inherent drawbacks and disadvantages which the present invention clearly addresses and resolves in a matter heretofore not taught.

It is therefore obvious that there is a great need for improvement in the field and that this is a problem which has been recognized but not effectively resolved within the known prior art.

### SUMMARY OF THE INVENTION

It is an object of the present invention to provide a chemical composition which can be used as an air filter for freshening the air resultant from substantially any type of contaminant. Such as the chemical composition can be used for eliminating odors emitted from litter boxes, laundry hampers, diaper pales, shoes, carpets, refrigerators and freezers, etc. Thus, it is to be understood this product can be used for any purpose according to user choice. Therefore, being such uses are much too numerous to list herein it is to be understood the present invention is not to be herein limited to any specific type of use.

It is another object of the present invention to provide a chemical composition which can be used as an air filter to maintain proper humidity levels to prolong the life of cherished personal belongings, such as gun collections, coin collections, stamp collections, etc.

Yet another object of the present invention is to provide a chemical composition which can be used as an air filter and which is made from natural minerals known as zeolites, which can be used alone or in combination with activated charcoal.

It is another object of the present invention to provide a chemical composition which can be used as an air filter that is exceptionally useful for preservation of food products. For example, if the present invention is used within a refrigerator, the refrigerator on an average stays 2-5 degrees colder, thereby creating a safer and fresher food storage environment whereby food stays fresher longer.

Yet another object of the present invention is to provide a chemical composition which when used as an air filter within a refrigerator reduces excess moisture and latent heat from the air, whereby the compressor runs less, thus saving energy and reducing the wear and tear on the refrigerator equipment.

Still a further object of the present invention is to provide a chemical composition that can be used as an air filter within a refrigerator to maintain proper humidity levels through the process known as adsorption (the reversible process of absorption and desorption). Whereby helping to inhibit the growth of bacteria, yeast and mold. Also, reducing excess moisture extends the life of the refrigerator by reducing structural deterioration from moisture damage.

Yet another object of the present invention is to provide a chemical composition that can be used as an air filter within a refrigerator and also because of reduced moisture, the air quality within the refrigerator is drier and crisper. Thus, the food stays fresher and lasts longer, whereby the consumer saves money and time as they need not shop as often.

Still a very important object and advantage which has not been recognized within the known prior art, is to provide a chemical composition that can be used as an air filter and as an aeration means. For example, after it has lost its effectiveness as an air filter, it can then be used as an extremely good aeration means for delivering oxygen to the roots of plants, by simply mixing the composition with the soil, such as within a garden area or potted plants.

Yet another object of the present invention is to provide a chemical composition that can be used as an air filter and we also provide a unique container for dispersing the product therefrom. Also, due to the novel hexagon shape of the container, this insures maximum airflow around the container, and also the container includes a sturdy “TYVEK” filter bag that allows water vapor to pass through easily. Also the contents are 100% natural which is very important.

Other objects and advantages will be seen when taken into consideration with the following specification and drawings.

### BRIEF DESCRIPTION OF THE DRAWINGS

FIG. 1 substantially represents an overview of the preferred embodiment for a container used in connection with the chemical composition of the present invention.

FIG. 2 is substantially a graph showing how the present invention greatly inhibits the natural growth of yeast and mold when the present invention is used with a food product, such as lettuce.

FIG. 3 is substantially a graph showing how the present invention greatly inhibits the natural growth of bacteria when the present invention is used with a food product, such as strawberries.

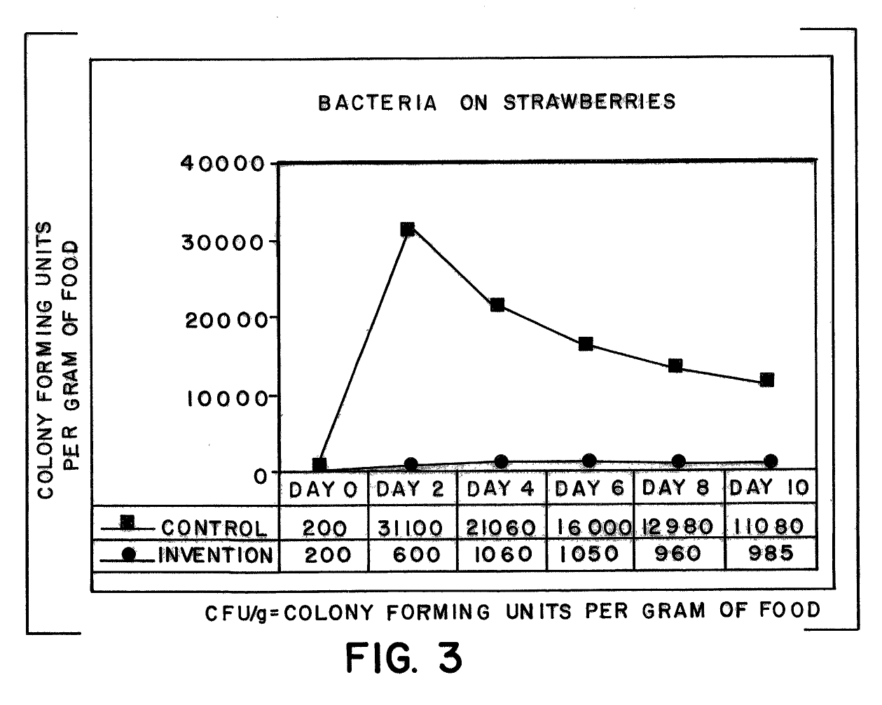

### DETAILED DESCRIPTION OF THE DRAWINGS

Referring now in detail to the drawings wherein like characters refer to like elements throughout the various views.

The present invention is substantially an air filter which is usable for freshening the environmental air surrounding the air filter, and can be manually positioned at any suitable location of user choice.

It is to be understood that the present invention is essentially a special blend of air freshening chemicals and the container for dispersing said chemicals can be any suitable container of manufacturing choice. Thus, the following description for the container is only exemplary of one suitable embodiment and therefore the invention is not to be limited to such a container.

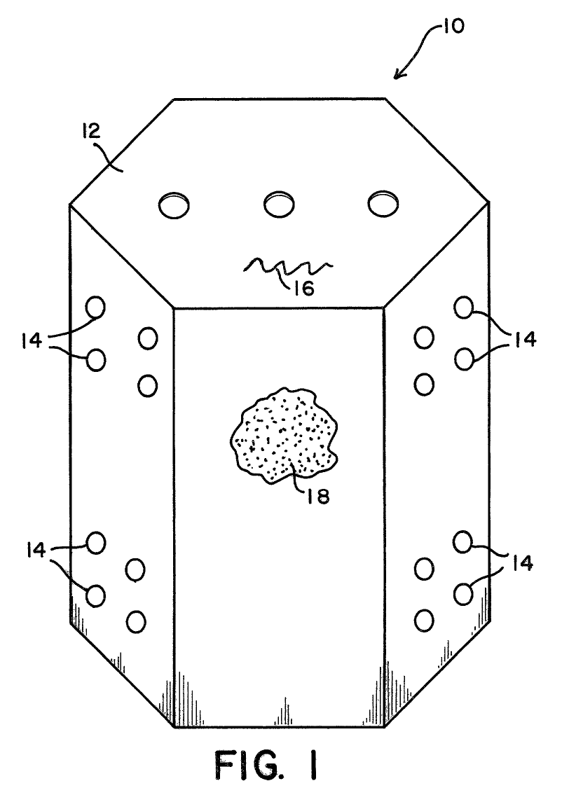

Referring now to FIG. 1, wherein (10) substantially represents an overview for the preferred embodiment for the present invention. As depicted therein, the air filter (10) of the present invention is formed from an air permeable container (12) which can be made from any suitable material of manufacturing choice, but the applicants prefer an environmentally safe, non-toxic material such as cardboard, or the like.

It is to be also understood the container (12) is preferably of a shape and size which facilitates proper dispersal of the air filter therefrom. For example as depicted herein, container (12) is hexagonal in shape and is substantially 5″ tall by 4″ wide, and further includes multiple aeration holes (14) which are appropriately spaced apart at intervals which allow for complete aeration throughout container (10).

Container (12) may also include pertinent indicia thereon of manufacturing choice, such as a logo, instructions, contents, etc., or as depicted herein, indicia (16) provides a space which allows the user to manually write in the date which informs them as to when the filter was opened. This is most useful as the user can easily determine from the date when it is time to replace the air filter.

Container (12) further an interior compartment which is of a shape and size to easily contain a filter bag (18). Thus, filter bag (18) is housed within air permeable container (12) and the filter bag has a chemical composition therein. It is to be understood any suitable type of filter may be used according to manufacturing choice, but the applicants contend a “TYVEK” filter bag (18) is most appropriate and functions to allow water vapor to easily pass there through.

As previously stated, filter bag (18) contains a chemical composition therein. It is to be understood the chemical composition in the preferred embodiment is substantially in particulate or granule form, but is not to be limited thereto. Also, it is to be further understood the chemical composition may be made from different ingredients of engineering choice, thus the following compositions are only exemplary of some suitable combinations.

The noted chemical composition may be 100% clinoptilolite. Or the chemical composition may be a combination of clinoptilolite with charcoal, such as 99% clinoptilolite and 1% charcoal. Or the chemical composition may vary between 95% to 99% clinoptilolite and between 1% to 5% charcoal.

Yet other suitable ingredients for producing the chemical composition includes a combination of clinoptilolite with chabazite, such as 99% clinoptilolite and 1% chabazite, or between 95% to 99% clinoptilolite and between 1% and 5% chabazite.

Yet another suitable chemical composition may be made from a combination of clinoptilolite, charcoal and chabazite, such as 98% clinoptilolite, 1% charcoal and 1% chabazite. Or another suitable combination may be between 99% clinoptilolite, ½% charcoal and ½% chabazite.

Or still a further combination may be between 96% to 99% clinoptilolite, between 1% to 2% charcoal, and between 1% to 2% chabazite.

Yet another suitable combination includes the chemical composition being any type of an odor absorbing zeolite. Such as 100% chabazite, or a combination of chabazite and charcoal, or still another suitable combination would include a combination of chabazite, clinoptilolite and charcoal, with the largest percentage being chabazite.

Other examples include a combination of an odor absorbing zeolite and charcoal in varying percentages with a greater amount of zeolite than charcoal. Such as 98% chabazite and 1% charcoal, or between 95% to 99% chabazite and between 1% and 5% charcoal.

Still another suitable chemical composition would include a combination clinoptilolite, charcoal and chabazite, such as 98% chabazite, 1% charcoal and 1% clinoptilolite. Or still further, a combination comprising 99% chabazite, ½% charcoal and ½% clinoptilolite. Also, another suitable combination would be 96% to 99% chabazite, between 1% to 2% charcoal, and between 1% to 2% clinoptilolite.

It is to be understood the present invention further teaches that the air filter can be considered to be a multi-purpose air filter. For example, the applicants have discovered that the air permeable container (12) with the filter bag (18), and the chemical composition in combination function as an air filter for a partial length of time, which is considered to be its first use.

Thereafter, the chemical composition is usable as an aeration means when the chemical composition is added to ground soil after the noted partial length of time has expired. Thus, the air filter has a second use as an aeration means for delivering oxygen to the roots of growing plants, and this is a very important function of the present invention.

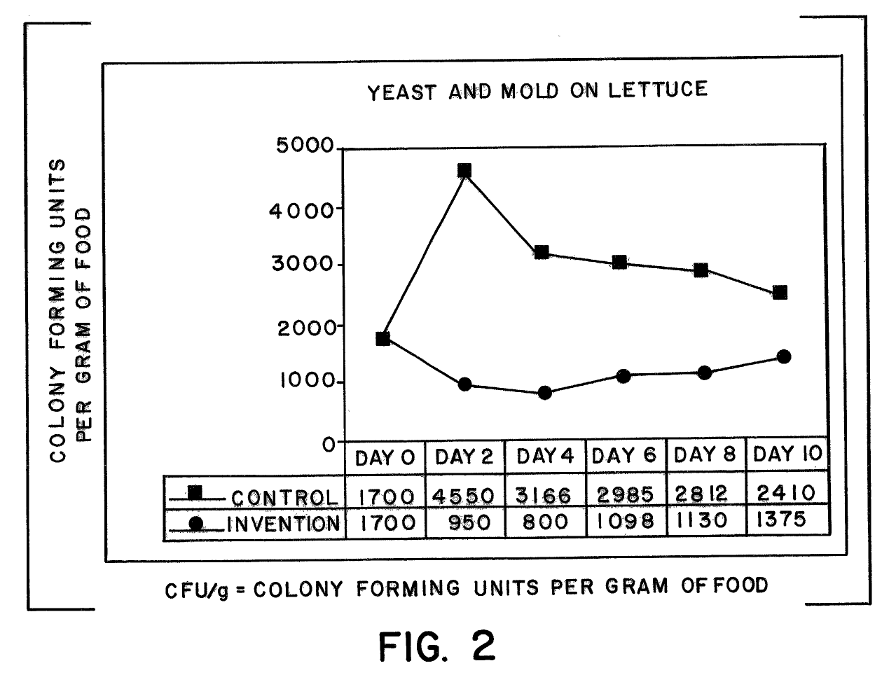

Referring now to FIGS. 2 & 3 which substantially represent the results of our experiments (which were conducted by “Tahoe food technologies”) when the air filter (10) of the present invention is used with a first food product such as lettuce, see FIG. 1, and a second food product such as strawberries, see FIG. 2.

It is to be understood such experiments as illustrated herein are simply depicted for informational purposes and being the illustrations are in graph form, the applicants contend we need not explain in any further detail as the graphs are self-explanatory.

It can now be seen we have herein presented an air fresher (10) which includes a unique container (12) and a filter (18) for dispensing the chemical composition therefrom, and now we also provide a method of use as follows:

A method for using an air filter (10), with the air filter being formed from an air permeable container (12) with a filter bag (18) and a chemical composition in combination. With the filter bag (18) being housed within air permeable container (12), and filter bag (18) having the noted chemical composition therein, comprising the steps of:

a. removing a protective outer wrap from the container (12);

b. positioning the container (12) centrally within an area wherein the surrounding environmental air is to be freshened;

c. leaving container (12) in position for a length of time, such as between 1 to 4 months; and;

d. removing container (12) from the noted area.

The method as described above further includes additional steps for proper environmentally safe disposal of air filter (10), comprising the steps of:

a. grasping container (12);

b. opening container (12);

c. opening filter bag (18); and;

d. mixing the noted chemical composition with the soil of an area which is to be used for growing plants;

whereby:

the noted chemical composition functions as aeration means for delivering oxygen to the roots of growing plants.

Although the invention has been shown and described in what is conceived to be the most practical and preferred embodiment, it is recognized that departures may be made therefrom within the scope and spirit of the invention, which is not to be limited to the details disclosed herein but is to be accorded the full scope of the claims so as to embrace any and all equivalent devices and apparatuses.

What we claim as new and wish to secure by Letters Patent is:

1. An air filter comprising: an air permeable container; a filter bag; and a chemical composition; said filter bag being housed within said air permeable container, said filter bag having said chemical composition therein, and said chemical composition being 100% clinoptilolite.

2. An air filter comprising: an air permeable container; a filter bag; and a chemical composition; said filter bag being housed within said air permeable container, said filter bag having said chemical composition therein, and said chemical composition being a combination of clinoptilolite with charcoal.

3. The air filter of claim 2 wherein said combination is 99% clinoptilolite and 1% charcoal.

4. The air filter of claim 2 wherein said combination varies between 95% to 99% clinoptilolite and between 1% to 5% charcoal.

5. An air filter comprising: an air permeable container; a filter bag; and a chemical composition; said filter bag being housed within said air permeable container, said filter bag having said chemical composition therein, and said chemical composition being a combination of clinoptilolite with chabazite.

6. The air filter of claim 5 wherein said combination is 99% clinoptilolite and 1% chabazite.

7. The air filter of claim 5 wherein said combination varies between 95% to 99% clinoptilolite, and between 1% to 5% chabazite.

8. An air filter comprising: an air permeable container; a filter bag; and a chemical composition; said filter bag being housed within said air permeable container, said filter bag having said chemical composition therein, and said chemical composition being a combination of clinoptilolite, charcoal and chabazite.

9. The air filter of claim 8 wherein said combination is 98% clinoptilolite, 1% charcoal and 1% chabazite.

10. The air filter of claim 8 wherein said combination is 99% clinoptilolite, ½% charcoal and ½% chabazite.

11. The air filter of claim 8 wherein said combination varies between 96% to 99% clinoptilolite, between 1% to 2% charcoal, and between 1% to 2% chabazite.

12. A multi-purpose air filter comprising: an air permeable container; a filter bag; and a chemical composition; said filter bag being housed within said air permeable container, and said filter bag having said chemical composition therein,
whereby:
said air permeable container, said filter bag; and
said chemical composition in combination function as an air filter for a partial length of time, and said chemical composition being usable as aeration means when said chemical composition is added to ground soil after said partial length of time.

13. A method for using an air filter, with said air filter being an air permeable container; a filter bag; and a chemical composition in combination; said filter bag being housed within said air permeable container, and said filter bag having said chemical composition therein, comprising the steps of:
a. removing a protective outer wrap from the container;
b. positioning said container centrally within an area wherein the surrounding environmental air is to be freshened;
c. leaving said container in position for a length of time between 1 to 4 months; and;
d. removing said container from said area.

14. The method of claim 13 further includes additional steps for proper environmentally safe disposal of said air filter, comprising the steps of:
a. grasping said container;
b. opening said container;
c. opening said filter bag; and;
d. mixing said chemical composition with soil of an area which is to be used for growing plants;
whereby:
said chemical composition functions as aeration means for delivering oxygen to the roots of said plants.

15. An air filter comprising: an air permeable container; a filter bag; and a chemical composition; said filter bag being housed within said air permeable container, said filter bag having said chemical composition therein, and said chemical composition being 100% of an odor absorbing zeolite.

16. An air filter comprising: an air permeable container; a filter bag; and a chemical composition; said filter bag being housed within said air permeable container, said filter bag having said chemical composition therein, and said chemical composition being a combination of an odor absorbing zeolite and charcoal.

17. The air filter of claim 16 wherein said combination of an odor absorbing zeolite and charcoal includes a greater amount of said odor absorbing zeolite than said charcoal.
